(19) United States
(12) Patent Application Publication
(10) Pub. No.: US 2021/0030909 A1
(43) Pub. Date: Feb. 4, 2021

(54) SYSTEMS FOR MONITORING AND CONTROLLING DISINFECTION

(71) Applicant: MOBILE UV INNOVATIONS PTY LTD, Victoria (AU)

(72) Inventor: Murray MCDONALD, Victoria (AU)

(21) Appl. No.: 16/981,946

(22) PCT Filed: Mar. 20, 2019
(86) PCT No.: PCT/AU2019/050242

§ 371 Date: Sep. 17, 2020

(30) Foreign Application Priority Data
Mar. 20, 2018 (AU) 2018900910

(51) Int. Cl. A61L 2/28; A61L 2/10; H04W 4/021; H04W 4/80; H04W 4/029

(52) U.S. Cl. CPC A61L 2/28; A61L 2/10; H04W 4/021; H04W 4/80; A61L 2202/11; A61L 2202/14; A61L 2202/25; A61L 2202/16; H04W 4/029

(57) ABSTRACT

A disinfection monitoring system, comprising: a sensor module configured to detect use of one or more disinfectants on a surface or in an area; a data logger module connected to the sensor module and configured to log data relating to detected uses of the one or more disinfectants; and a wireless communication module connected to the data logger module and configured to wirelessly communicate the logged data relating to detected use of the one or more disinfectants to a remote monitoring module.

### FIELD

The present invention relates to systems for monitoring and controlling disinfection in hospitals, healthcare facilities, aged care facilities, specialist offices, medical centres, research labs, veterinary labs, childcare facilities, schools, etc.

### BACKGROUND

Hospital-acquired infections (HAIs) kill many patients every year and contribute to rising healthcare costs. HAIs may be reduced or avoided by disinfecting surfaces and areas in hospitals using chemical disinfectants, such as bleach or hydrogen peroxide vapour. In addition, germicidal ultraviolet (UV) light in the C spectrum (UVC) is starting to be used to disinfect hospital surfaces, equipment, instruments, rooms and areas.

However, because of time demands and human errors which frequently occur in hospitals, health care providers or hospital cleaners may forget or skip disinfection treatments, or only perform minimal disinfection treatments on an occasional basis.

In this context, there is a need for improved solutions for monitoring and controlling disinfection.

### SUMMARY

According to the present invention, there is provided a disinfection monitoring system, comprising:

a sensor module configured to detect use of one or more disinfectants on a surface or in an area;

a data logger module connected to the sensor module and configured to log data relating to detected uses of the one or more disinfectants; and

a wireless communication module connected to the data logger module and configured to wirelessly communicate the logged data relating to detected use of the one or more disinfectants to a remote monitoring module.

The one or more disinfectants may be selected from a light-based disinfectant, a gas disinfectant, a vapour disinfectant, a liquid disinfectant, and combinations thereof.

The light-based disinfectant may be UV light, the vapour disinfectant may be hydrogen peroxide vapour, and the liquid disinfectant may be bleach.

The sensor module may comprise one or more sensors selected from a UV light sensor, a gas sensor, a vapour sensor, a chemical sensor, a thermal sensor, and combinations thereof.

The sensor module may further comprise a location sensor, such as a global positioning system (GPS) sensor.

The data logger module may comprise a data storage device configured to store the logged data relating to detected use of the one or more disinfectants.

The wireless communication module may comprise a low power wireless communication module, such as a Bluetooth low power module.

The remote monitoring module may be a cloud-based monitoring and reporting application, such as a cloud dashboard.

The disinfection monitoring system may further comprise a microcontroller connected to a power source and each of the sensor module, the data logger module and the wireless communication module.

The disinfection monitoring system may further comprise a visual indicator connected to the microcontroller and the power source, wherein the microcontroller is configured to control the visual indicator to visually indicate the disinfection status of the surface or the area.

The visual indicator may be a light emitting diode (LED) indicator.

The power source may be a battery, a solar panel, or both.

The present invention also provides a UV disinfection system, comprising:

a UV lamp configured to apply UV light to a surface or an area in a hospital;

a mobile base configured to adjustably support the UV lamp adjacent to the surface or the area; and

a controller connected to the UV lamp and configured to control the UV lamp to vary the UV light applied to the surface or the area.

The UV disinfection system may further comprise:

a UV light sensor connected to the controller and configured to detect the UV light applied to the surface or the area;

wherein the controller is further configured to control the UV lamp and vary the UV light applied to the surface or the area based on the UV light detected by the UV light sensor.

The UV light sensor of the UV disinfection system may be the UV light sensor of the disinfection monitoring system described above.

The UV disinfection system may further comprise:

a location sensor connected to the controller and configured to detect the location of the UV lamp;

wherein the controller is further configured to control the UV lamp and vary the UV light applied to the surface or the area based on the location of the UV lamp detected by the location sensor.

The UV disinfection system may further comprise:

a distance measuring sensor connected to the controller and configured to detect the distance between the UV lamp and the surface or the area;

wherein the controller is further configured to control the UV lamp and vary the UV light applied to the surface or the area based on the distance between the UV lamp and the surface or the area detected by the distance measuring sensor.

The UV disinfection system may further comprise a wireless remote controller connected to the controller.

### BRIEF DESCRIPTION OF DRAWINGS

Embodiments of the invention will now be described by way of example only with reference to the accompanying drawings, in which:

FIG. 1 is a block diagram of an example embodiment of a disinfection monitoring system according to an example embodiment of the present invention;

FIG. 2 is a schematic diagram of an example LED indicator of the disinfection monitoring system of FIG. 1;

FIG. 3 is a block diagram of an example embodiment of a UV disinfection system according to an example embodiment of the present invention; and

FIG. 4 is a computer rendering of the hospital monitoring UV disinfection system of FIG. 3.

### DESCRIPTION OF EMBODIMENTS

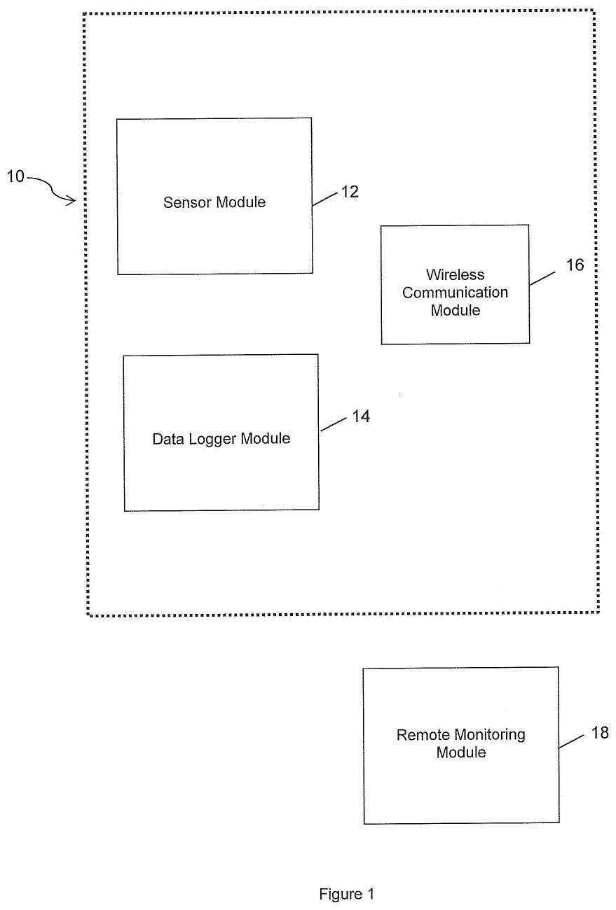

Referring to FIG. 1, an example disinfection monitoring system 10 according to an example embodiment of the present invention may generally comprise a sensor module 12, a data logger module 14, and a wireless communication module 16.

The sensor module 12 may be configured to detect use of one or more disinfectants on a surface or in an area in hospitals, healthcare facilities, aged care facilities, specialist offices, medical centres, research labs, veterinary labs, childcare facilities, schools, etc. The sensor module 12 may be configured to detect the presence or absence of the one or more disinfectants on the surface or in the area. The one or more disinfectants may be selected from a light-based disinfectant, a gas disinfectant, a vapour disinfectant, a liquid disinfectant, and combinations thereof. The sensor module 12 may therefore comprise one or more sensors selected from a UV light sensor, a gas sensor, a vapour sensor, a chemical sensor, a thermal sensor, and combinations thereof.

For example, the light-based disinfectant may be UVC light, the vapour disinfectant may be hydrogen peroxide vapour, and the liquid disinfectant may be bleach. In one example embodiment, the sensor module 12 may therefore comprise a UV light sensor, a hydrogen peroxide vapour sensor (eg, a near infrared (NIR) sensor), and a chemical sensor capable of detecting bleach (or chemical constituents thereof).

The sensor module 12 may further comprise a location sensor, such as a GPS sensor. This may enable the location of the sensor module 12 within the hospital to be monitored and tracked.

The data logger module 14 may be connected to the sensor module 12 and configured to log data relating to detected uses of the one or more disinfectants. The data logger module 14 may comprise a data storage device configured to store the logged data relating to detected use of the one or more disinfectants. The logged data may comprise date, time, location, level of disinfectant present, etc.

The wireless communication module 16 may be connected to the data logger module 14 and configured to wirelessly communicate the logged data relating to detected use of the one or more disinfectants to a remote monitoring module 18. The wireless communication module may comprise a low power wireless communication module, such as a Bluetooth low power module. The remote monitoring module 18 may be a cloud-based monitoring and reporting application, such as a cloud dashboard. The cloud dashboard may provide monitoring, tracking, analytics, alerts, and reporting of the disinfection status of individual surfaces or areas in hospitals.

The disinfection monitoring system 10 may further comprise a microcontroller (not shown) connected to a power source (not shown) and each of the sensor module 12, the data logger module 14 and the wireless communication module 16. The power source may be a battery (not shown), a solar panel (not shown), or both.

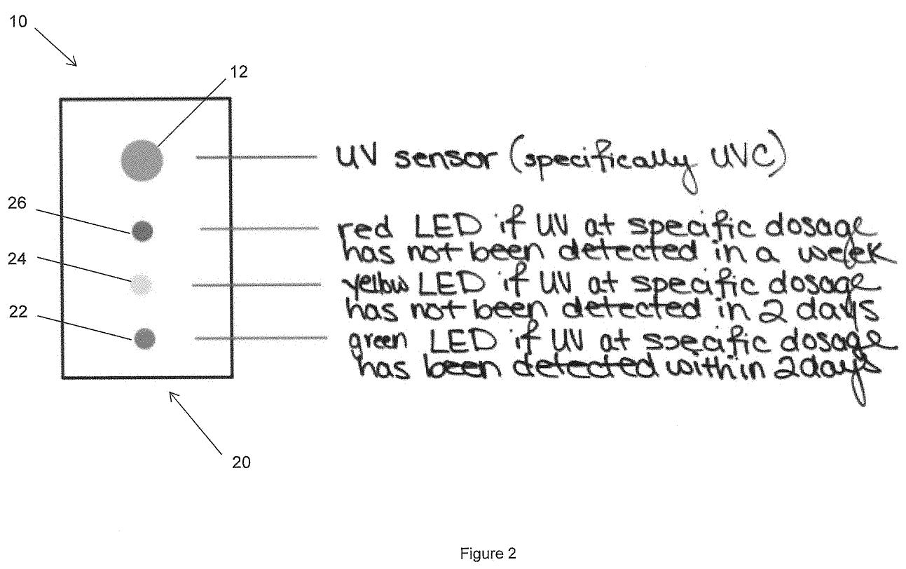

Referring to FIG. 2, a visual indicator 20 may be connected to the microcontroller and the power source, and the microcontroller may be configured to control the visual indicator 20 to visually indicate the disinfection status of the surface or the area. The visual indicator 20 may, for example, be a traffic-light LED indicator 20 having green, yellow and red LEDs 22, 24, 26. For example, a green LED 22 may visually indicate that disinfection is not required, while yellow or red LEDs 24, 26 may visually indicate that disinfection is required.

The sensor module 12, the data logger module 14 and the wireless communication module 16, power source, microcontroller, and traffic-light LED indicator 20 may be provided together on a printed circuit board (not shown), and housed together in a common housing (not shown) that may be removably attachable or positionable on individual surfaces or in individual areas of hospitals, A plurality of the disinfection monitoring systems 10 may be provided as a network of disinfection sensors in hospitals, healthcare facilities, aged care facilities, specialist offices, medical centres, research labs, veterinary labs, childcare facilities, schools, etc.

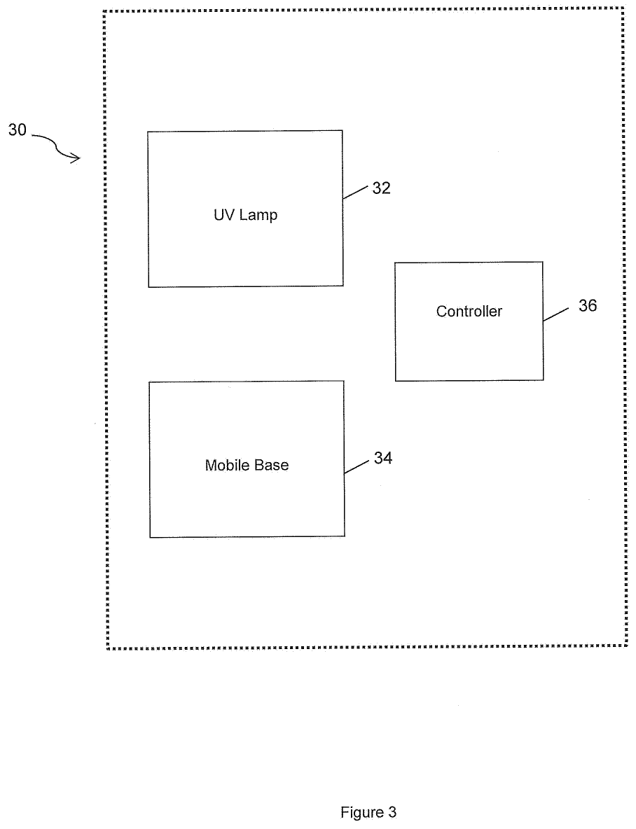

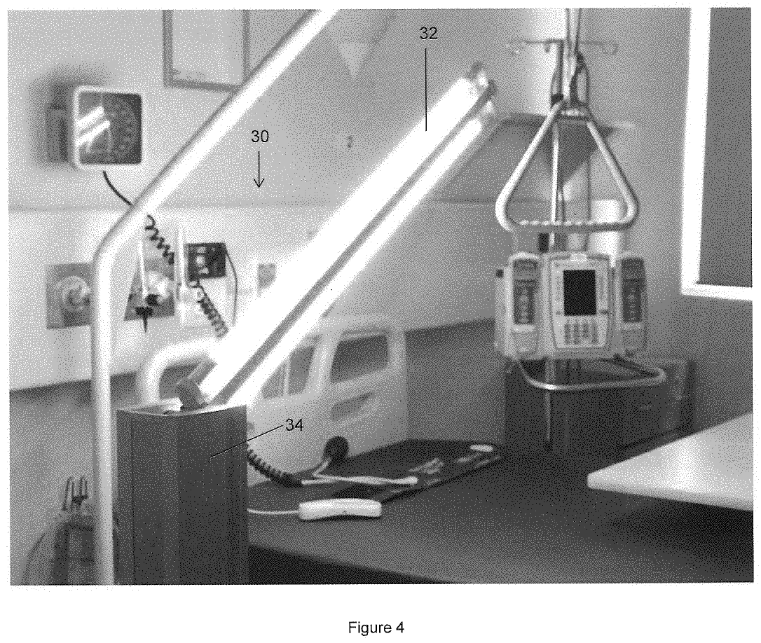

Referring to FIG. 3, an example UV disinfection system 30 according to an example embodiment of the present invention generally comprises a UV lamp 32, a mobile base 34 and a controller 36. The UV lamp 32 may be configured to apply UV light, for example UVC light, to a surface or an area in a hospital. The UV lamp 32 may comprise a plurality of UV lamps 32. The mobile base 34 may be configured to adjustably support the UV lamp 32 adjacent to the surface or the area. For example, as illustrated in FIG. 4, the mobile base 34 may, for example, be a wheeled cabinet that pivotally adjustably supports two UV lamps 32. The controller 36 may be connected to the UV lamp 32 and configured to control the UV lamp 32 to vary the UV light applied to the surface or the area.

The UV disinfection system 30 may further comprise a UV light, sensor (not shown) connected to the controller 36 and configured to detect the UV light applied to the surface or the area. The controller 36 may be further configured to control the UV lamp 32 and vary the UV light applied to the surface or the area based on the UV light detected by the UV light sensor. The UV light sensor of the UV disinfection system may, for example, be the UV light sensor of the disinfection monitoring system 10 described above.

The UV disinfection system 30 may further comprise a location sensor (not shown) connected to the controller 36 and configured to detect the location of the UV lamp 32 or the mobile base 34. The controller 34 may be further configured to control the UV lamp 32 and vary the UV light applied to the surface or the area based on the location of the UV lamp 32 or the mobile base 34 detected by the location sensor.

The UV disinfection system 30 may further comprise a distance measuring sensor connected to the controller 36 and configured to detect the distance between the UV lamp 32 and the surface or the area. The controller 36 may be further configured to control the UV lamp 32 and vary the UV light applied to the surface or the area based on the distance between the UV lamp 32 and the surface or the area detected by the distance measuring sensor.

The UV disinfection system 30 may further comprise a wireless remote controller connected to the controller 30 to enable remote operation of the UV lamp 32. The usage, location, and UV dosage of the UV disinfection system 30 may be monitored, tracked, analysed and reported using the disinfection monitoring system 10 described above.

Embodiments of the present invention provide systems that are both generally and specifically useful for monitoring disinfection of surfaces, equipment, instruments, rooms and areas in hospitals, healthcare facilities, aged care facilities, specialist offices, medical centres, research labs, veterinary labs, childcare facilities, schools, etc. In addition, other embodiments, the present invention provides systems that are both generally and specifically useful for UV disinfection of surfaces, equipment, instruments, rooms and areas in hospitals, healthcare facilities, aged care facilities, specialist offices, medical centres, research labs, veterinary labs, childcare facilities, schools, etc.

For the purpose of this specification, the word “comprising” means “including but not limited to,” and the word “comprises” has a corresponding meaning.

The above embodiments have been described by way of example only and modifications are possible within the scope of the claims that follow.

1. A disinfection monitoring system, comprising:
a sensor module configured to detect use of one or more disinfectants on a surface or in an area;
a data logger module connected to the sensor module and configured to log data relating to detected uses of the one or more disinfectants; and
a wireless communication module connected to the data logger module and configured to wirelessly communicate the logged data relating to detected use of the one or more disinfectants to a remote monitoring module.

2. The system of claim 1, wherein the one or more disinfectants are selected from a light-based disinfectant, a gas disinfectant, a vapour disinfectant, a liquid disinfectant, and combinations thereof.

3. The system of claim 2, wherein the light-based disinfectant is UV light, the vapour disinfectant is hydrogen peroxide vapour, and the liquid disinfectant is bleach.

4. The system of claim 2, wherein the sensor module comprises one or more sensors selected from a UV light sensor, a gas sensor, a vapour sensor, a chemical sensor, a thermal sensor, and combinations thereof.

5. The system of claim 4, wherein the sensor module further comprises a location sensor.

6. The system of claim 5, wherein the location sensor is a global positioning system (GPS) sensor.

7. The system of claim 1, wherein the data logger module comprises a data storage device configured to store the logged data relating to detected use of the one or more disinfectants.

8. The system of claim 1, wherein the wireless communication module comprises a low power wireless communication module.

9. The system of claim 8, wherein the low power wireless communication module is a Bluetooth low power module.

10. The system of claim 1, wherein the remote monitoring module is a cloud-based monitoring and reporting application.

11. The system of claim 10, wherein the cloud-based monitoring and reporting application is a cloud dashboard.

12. The system of claim 1, further comprising a microcontroller connected to a power source and each of the sensor module, the data logger module and the wireless communication module.

13. The system of claim 12, further comprising a visual indicator connected to the microcontroller and the power source, wherein the microcontroller is configured to control the visual indicator to visually indicate the disinfection status of the surface or the area.

14. The system of claim 13, wherein the visual indicator is a Sight emitting diode (LED) indicator.

15. The system of claim 12, wherein the power source is a battery, a solar panel, or both.

16. A ultraviolet (UV) disinfection system, comprising:
a UV lamp configured to apply UV light to a surface or an area in a hospital;
a mobile base configured to adjustably support the UV lamp adjacent to the surface or the area; and
a controller connected to the UV lamp and configured to control the UV lamp to vary the UV light applied to the surface or the area.

17. The system of claim 16, further comprising:
a UV light sensor connected to the controller and configured to detect the UV light applied to the surface or the area;
wherein the controller is further configured to control the UV lamp and vary the UV light applied to the surface or the area based on the UV light detected by the UV light sensor.

18. The system of claim 17, wherein the UV light sensor includes one or more sensors selected from the UV light sensor, a gas sensor, a vapour sensor, a chemical sensor, a thermal sensor, and combinations thereof.

19. The system of claim 18, further comprising:
a location sensor connected to the controller and configured to detect the location of the UV lamp;
wherein the controller is further configured to control the UV lamp and vary the UV light applied to the surface or the area based on the location of the UV lamp detected by the location sensor.

20. The system of claim 16, further comprising:
a distance measuring sensor connected to the controller and configured to detect the distance between the UV lamp and the surface or the area;
wherein the controller is further configured to control the UV lamp and vary the UV light applied to the surface or the area based on the distance between the UV lamp and the surface or the area detected by the distance measuring sensor.

21. The system of claim 16, further comprising a wireless remote controller connected to the controller.
